(19) United States
(12) Patent Application Publication
(10) Pub. No.: US 2026/0360540 A1
(43) Pub. Date: Nov. 12, 2026

(54) DEPRESSION PREDICTION DEVICE AND METHOD BASED ON NUTRITIONAL FACTORS USING EXPLAINABLE ARTIFICIAL INTELLIGENCE

(71) Applicants: CHA UNIVERSITY INDUSTRY-ACADEMIC COOPERATION FOUNDATION, Pocheon-si (KR); SUNGKWANG MEDICAL FOUNDATION, Seoul (KR)

(72) Inventors: Myung-Gwan Kim, Seongnam-si (KR); HyunWook Han, Seongnam-si (KR); JinHyung Lee, Anyang-si (KR); Joon Ho Park, Incheon (KR)

(73) Assignees: CHA UNIVERSITY INDUSTRY-ACADEMIC COOPERATION FOUNDATION, Pocheon-si (KR); SUNGKWANG MEDICAL FOUNDATION, Seoul (KR)

(21) Appl. No.: 19/437,311

(22) Filed: Dec. 31, 2025

Related U.S. Application Data
(63) Continuation of application No. PCT/KR2024/096886, filed on Dec. 13, 2024.

(30) Foreign Application Priority Data
Nov. 22, 2024 (KR) 10-2024-0168348

(51) Int. Cl. G16H 50/20; G16H 10/60; G16H 20/60

(52) U.S. Cl. CPC G16H 50/20; G16H 10/60; G16H 20/60

(57) ABSTRACT

The present invention relates to a depression prediction device and method based on nutritional factors using explainable artificial intelligence, wherein the depression prediction device based on nutritional factors includes a data collection unit configured to obtain variable data for each of multiple nutritional factors analyzed from a dietary status of a target patient; and a classification unit configured to classify a depression level and a normality state of the target patient by inputting the variable data for each of the multiple nutritional factors to a pre-learned depression level prediction model.

### CROSS REFERENCE TO PRIOR APPLICATIONS

This application is a continuation application of PCT International Patent Application No. PCT/KR2024/096886 filed on Dec. 13, 2024, which claims priority to Korean Patent Application No. 10-2024-0168348 filed on Nov. 22, 2024, which are all hereby incorporated by reference in their entirety.

### ACKNOWLEDGEMENT

This research was supported by the Institute for Information & Communications Technology Planning & Evaluation (IITP) grant funded by the Ministry of Science and ICT (No. 2710033981).

### BACKGROUND

The present invention relates to depression prediction device and method based on nutritional factors using explainable artificial intelligence, and more particularly, to depression prediction device and method that can predict depression severity level and normality of a patient from the patient's nutritional factors based on explainable artificial intelligence and predict depression based on the patient's nutritional factors using the explainable artificial intelligence.

The existing mental illness diagnosing method ultimately determine what type of mental illness a patient has by relying on the knowledge and experience of psychiatrists, based on the interpretation of results of a standardized questionnaire and the complaints of patients with mental illness.

However, despite fragmentary research demonstrating that various nutrients ingested by the body can influence mental health conditions such as depression, the research is not sufficiently considered in clinical practice.

Furthermore, while the number of patients with mental illness is rapidly increasing, examining patient's nutritional states at every visit and providing personalized precision medicine are difficult without the help of artificial intelligence technology.

Through the development of specialized artificial intelligence systems, a platform that can predict depression severity levels by considering a patient's nutritional factors is needed.

The technology underlying the present invention is disclosed in Korea Patent Publication No. 10-2023-0101328 (published on Jul. 6, 2023).

### SUMMARY

An object of the present invention is to provide a depression prediction device and method that can predict depression based on nutritional factors using explainable artificial intelligence and predict a depression level and normality by applying the nutritional factors of a patient to an artificial intelligence model.

According to the present invention, a depression prediction device based on nutritional factors includes a data collection unit configured to obtain variable data for each of multiple nutritional factors analyzed from a dietary status of a target patient; and a classification unit configured to classify a depression level and a normality state of the target patient by inputting the variable data for each of the multiple nutritional factors to a pre-learned depression level prediction model.

Also, the classification unit may classify the depression level of the target patient into one of a moderate level, a mild level, and a normal level.

Also, the data collection unit may generate variable data for each nutritional factor based on a nutrient intake state analyzed from a diet history of the target patient during a set period.

Also, the multiple nutritional factors may include a plurality of factors selected from n3-fatty acid, calcium, potassium, sodium, iron, vitamin B1, vitamin B3, vitamin C, monounsaturated fatty acid, retinol, carbohydrate, n3-fatty acid, n6-fatty acid, a water intake amount, cholesterol, dietary fiber, monounsaturated fatty acid, and saturated fatty acid.

Also, the depression prediction device may further include a provision unit configured to analyze the variable data for each of the multiple nutritional factors of the target patient input to the depression level prediction model, and presents analysis content of impact for each factor on which nutritional factor causes a classification result of a depression level for a corresponding patient to be derived, by using explainable artificial intelligence.

Also, the data collection unit may further obtain demographic information including gender and age of the target patient, and the classification unit may classify a depression severity level and a normal state by determining differently a combination of nutritional factors to be applied to the depression level prediction model depending on the gender and age of the target patient.

Also, the classification unit may classify a depression level and a normality state of a target patient who is male and aged between 20 and 39 years by utilizing variable values of factors corresponding to n3-fatty acid, calcium, sodium, iron, vitamin B3, vitamin C, monounsaturated fatty acid, retinol, carbohydrate, and n6-fatty acid among variable values for each nutritional factor collected for the target patient, and may classify a depression level and a normality state of a target patient who is female and aged between 20 and 39 years by utilizing variable values of factors corresponding to n6-fatty acid, vitamin C, beta-carotene, monounsaturated fatty acid, n3-fatty acid, dietary fiber, saturated fatty acid, carbohydrate, sodium, and potassium among variable values for each nutritional factor collected for the target patient.

Also, the classification unit may classify a depression level and a normality state of a target patient who is male and aged between 40 and 59 years by utilizing variable values of factors corresponding to a water intake amount, vitamin B1, carbohydrate, vitamin B3, beta-carotene, calcium, cholesterol, dietary fiber, potassium, and monounsaturated fatty acid among variable values for each nutritional factor collected for the target patient, and may classify a depression level and a normality state of a target patient who is female and aged between 40 and 59 years by utilizing variable values of factors corresponding to iron, dietary fiber, sodium, saturated fatty acid, n3-fatty acid, a water intake amount, calcium, vitamin B1, retinol, and n6-fatty acid among variable values for each nutritional factor collected for the target patient.

In addition, according to the present invention, a depression prediction method, which is performed by a depression prediction device based on nutritional factors, includes a step of obtaining variable data for each of multiple nutritional factors analyzed from a dietary status of a target patient; and normality state of the target patient by inputting the variable data for each of the multiple nutritional factors to a pre-learned depression level prediction model.

Also, the depression prediction method may further include a step of analyzing the variable data for each of the multiple nutritional factors of the target patient input to the depression level prediction model, and presenting analysis content of impact for each factor on which nutritional factor causes a classification result of a depression level for a corresponding patient to be derived, by using explainable artificial intelligence.

According to the present invention, by applying nutritional factors considering a patient's dietary state to an artificial intelligence model, depression levels and normality states can be predicted without the need for a medical professional's direct assessment.

Furthermore, by using an explainable artificial intelligence model, a medical professional can intuitively identify nutritional factors that influence severity classification, diagnostic work efficiency of the medical professional can be improved, and thus, the medical professional can focus on essential treatment of depression patients.

Therefore, the present invention can be utilized in precision medicine for prevention and treatment of depression by utilizing an explainable artificial intelligence technology considering nutritional factors.

### BRIEF DESCRIPTION OF THE DRAWINGS

FIG. 1 is a diagram illustrating a depression prediction system based on nutritional factors using explainable artificial intelligence, according to an embodiment of the present invention.

FIG. 2 is a diagram illustrating a configuration of a depression prediction device illustrated in FIG. 1.

FIG. 3 is a flowchart illustrating a depression prediction method according to an embodiment of the present invention.

FIG. 4 is an example diagram illustrating a learning data construction process according to an embodiment of the present invention.

FIG. 5 is an example diagram illustrating a depression prediction model learning and interpretation process considering nutritional factors, according to an embodiment of the present invention.

FIGS. 6A to 6D are example diagrams illustrating depression prediction results considering nutritional factors to which gender and age differences are reflected, according to an embodiment of the present invention.

### DETAILED DESCRIPTION OF THE INVENTION

Hereinafter, embodiments of the present invention will be described in detail with reference to the attached drawings such that those skilled in the art to which the present invention belongs can easily practice the embodiments. However, the present invention may be implemented in various different forms and is not limited to the embodiments described herein. In addition, for the purpose of clearly describing the present invention, parts irrelevant to the description are omitted in the drawings, and similar parts are denoted by similar reference numerals throughout the specification.

Throughout the specification, when a portion is described to be “connected” to another portion, this includes not only a case where the portion is “directly connected” thereto but also a case where the portion is “electrically connected” thereto with another element therebetween. Also, when a portion is described to “include” a component, this does not exclude other components, but rather includes other components, unless otherwise specified.

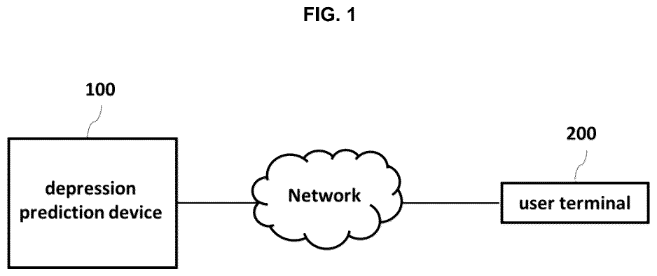

FIG. 1 is a diagram illustrating a depression prediction system based on nutritional factors using explainable artificial intelligence, according to an embodiment of the present invention.

As illustrated in FIG. 1, the depression prediction system according to an embodiment of the present invention may include a depression prediction device 100 and a user terminal 200.

The depression prediction device 100 according to an embodiment of the present invention can be connected to the user terminal 200 through a wired, wireless, or wired/wireless combined network and exchange information with each other. The wireless network may include at least one of radio RF, WLAN, Wi-Fi, and Bluetooth, and may utilize various known wireless network methods.

The depression prediction device 100 may be implemented as an online platform, such as a web server or app server, which provides an artificial intelligence-based depression level prediction service to the network-connected user terminal 200, or may be implemented in the user terminal or so on as an application program, an application, or so on.

The depression prediction system 100 may provide the network-connected user terminal 200 with a depression prediction service platform considering nutritional factors, implemented as an app or web. The service platform may be an application program being executed in an app or web environment.

In this way, the depression prediction system 100 can be implemented as an application program (application) being executed by a platform server or the user terminal 200 that provides a depression prediction service considering nutritional factors, and the user terminal 200 can receive related services by being connected to the device 100 through a network while the related application program is executed.

The user terminal 200 may include a device capable of exchanging information by connecting to a wired or wireless network, such as a PC, desktop, smart phone, tablet, or notebook. Here, the user terminal 200 may correspond to a medical staff terminal or a general user terminal.

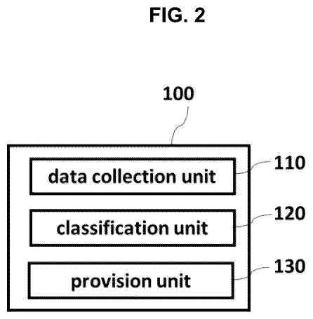

FIG. 2 is a diagram illustrating a configuration of the depression prediction device illustrated in FIG. 1.

As illustrated in FIG. 2, the depression prediction device 100 according to an embodiment of the present invention may include a data collection unit 110 and a classification unit 120, and may further include a provision unit 130 and a learning unit (not illustrated). Here, operations of the respective units 110 to 130 and a data flow between the respective units may be controlled by a control unit (not illustrated).

This depression prediction device 100 may be physically configured, and may also be implemented as a computer device including a processor, a memory, a user interface input/output device, a storage device, a network input/output device, and so on, or may also be implemented as an application program being executed by the computer device or a user terminal.

The data collection unit 110 may obtain variable data for multiple nutritional factors analyzed from a dietary state of a target patient.

Here, the multiple nutritional factors may include multiple factors selected from among n3-fatty acid, calcium, potassium, sodium, iron, vitamin B1, vitamin B3, vitamin C, monounsaturated fatty acid, retinol, carbohydrate, n3-fatty acid, n6-fatty acid, water intake amount, cholesterol, dietary fiber, monounsaturated fatty acid, and saturated fatty acid.

In this case, the data collection unit 110 may generate variable data for each nutritional factor based on a nutrient intake state analyzed from a diet history of a target patient for a set period.

The data collection unit 110 can grasp a nutrient intake state from a diet history of a target patient input from the user terminal 200 during a set period (for example, one week, two weeks, or one month) and generate variable data for each nutritional factor. For example, the data collection unit 110 can apply the dietary history for the set period to a pre-set nutritional analysis algorithm to quantify an expected intake amount for each nutritional factor during the set period.

Of course, the data collection unit 110 can also obtain numerical data for each nutritional factor, which is pre-analyzed or calculated from a target patient's dietary state, directly from the user terminal 200, and can also obtain related data from health and nutrition survey results conducted by a national or designated institution.

Next, the classification unit 120 may input the obtained variable data for each nutritional factor on the target patient to a pre-learned depression level prediction model to classify a depression level and normality of the target patient.

Through the depression level prediction model, the classification unit 120 can classify the target patient's depression level into one of a moderate level, a mild level, and a normal level. The depression level prediction model can be pre-learned by artificial intelligence and implemented by a deep learning-based intelligent algorithm.

The depression level prediction model can be pre-learned by the learning unit (not illustrated). The learning unit can train a depression classification model using variable values for each nutritional factor collected for multiple patients in the past and depression diagnosis scores or results. The multiple patients may include a normal patient group, a moderate depression patient group, and a mild depression patient group.

The learning unit can pre-train a depression level classification model by using variable values for each nutritional factors collected for multiple normal patients and depressed patients in the past as input data and using labeling values (for example, ‘1’=Moderate, ‘2’=Mild, ‘3’=Normal) corresponding to the medical team's diagnosis results for each patient as output data.

In addition to nutritional factors for patients, demographic information on patients may also be utilized for training a depression level prediction model. Demographic information may include age, gender, income, an education level, residence, the number of family members, a residential state, a marital state, BMI (body mass index), PHQ-9 (depression assessment tool) score, a medication use state, smoking, a drinking state, and so on.

Accordingly, by simply inputting an individual's state including gender, age, and a nutrient intake amount to a depression level prediction model, a depression level (for example, a severity level, mild depression probability, a normal state, or so on) can be predicted.

Next, the provision unit 130 can output and provide to the user terminal 200 the depression level and a depression state of a target patient classified by the depression level prediction model.

Furthermore, the provision unit 130 can analyze variable data for each nutritional factor of a target patient input to a depression level prediction model and may present analysis content (analysis result of the influence of each factor) of impact for each factor on which nutritional factor causes a classification result of the depression level for a corresponding patient to be derived, by using explainable artificial intelligence.

The present invention can provide factors that can be explained by an artificial intelligence model and can visualize factors that contribute to the severity depression probability in a personalized manner by comparing the factors to input values, and can consider elements whose importances are greater than a set value (for example, +0.2) as valid features. A standard for the influence of depression on the input nutrient intake amount is based on a recommended intake amount of the Korean nutrient intake standard (2020) presented by the Korean Nutrition Society. For example, when an average recommended intake amount of vitamin B3 (niacin) is approximately 15 mg and an input value is low at 0.18 mg and the importance of contributing to the probability of depression is +0.6, a low vitamin B3 intake amount can be interpreted as a factor that increases depression.

Thus, the present invention can provide an artificial intelligence-based depression prediction system that efficiently can predict and classify a depression level and a normality state by training nutritional factors derived from dietary states of depressed patients through artificial intelligence.

In an embodiment of the present invention, the data collection unit 110 can further obtain demographic information including gender and age of a target patient, and the classification unit 120 can determine different combinations of nutritional factors applied to the depression level prediction model based on the gender and age of the target patient, thereby classifying the severity of depression and a normal state.

More specifically, according to an embodiment of the present invention, patients are classified into four groups based on gender and age, and based on the results trained through artificial intelligence that can explain patient data for each group, top nutritional factors with high influence (for example, top 10 factors) can be identified, and the identified top factors can be utilized to optimize a depression level prediction model for each group.

For example, for a target patient of a first group who is male and aged between 20 and 39 years, the classification unit 120 can classify a depression level and a normality state of the target patient by utilizing variable values of factors corresponding to n3-fatty acid, calcium, sodium, iron, vitamin B3, vitamin C, monounsaturated fatty acid, retinol, carbohydrate, and n6-fatty acid, which are analyzed as top 10 priority nutrients, among variable values for each nutritional factor collected for the target patient.

Also, for a target patient of a second group who is male and aged between 40 and 59 years, the classification unit 120 can classify a depression level and a normality state of the target patient by utilizing variable values of factors corresponding to a water intake amount, vitamin B1, carbohydrate, vitamin B3, beta-carotene, calcium, cholesterol, dietary fiber, potassium, and monounsaturated fatty acid, which are analyzed as top 10 priority nutrients, among variable values for each nutritional factor collected for the target patient.

Also, for a target patient who is female and aged between 20 and 39 years, the classification unit 120 can classify a depression level and a normality state of the target patient by utilizing variable values of factors corresponding to n6-fatty acid, vitamin C, beta-carotene, monounsaturated fatty acid, n3-fatty acid, dietary fiber, saturated fatty acid, carbohydrate, sodium, and potassium, which are analyzed as top 10 priority nutrients, among variable values for each nutritional factor collected for the target patients.

Also, for a target patient who is female and aged between 40 and 59 years, the classification unit 120 can classify a depression level and a normality state of the target patient by utilizing variable values of factors corresponding to iron, dietary fiber, sodium, saturated fatty acid, n3-fatty acid, a water intake amount, calcium, vitamin B1, retinol, and n6-fatty acid, which are analyzed as top 10 priority nutrients among variable values for each nutritional factor collected for the target patient.

In this way, according to the present invention, depression levels and normality states can be predicted by utilizing combinations of factors selected differently depending on genders and ages of target patients.

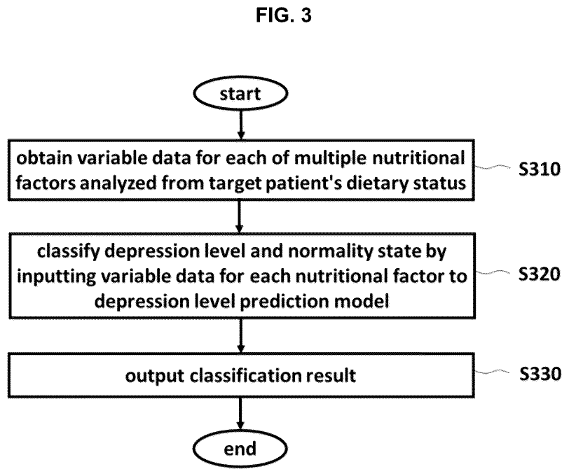

FIG. 3 is a diagram illustrating a depression prediction method according to an embodiment of the present invention.

First, the depression prediction device 100 obtains variable data for each of multiple nutritional factors analyzed from a target patient's dietary status (S310).

In addition, the depression prediction device 100 can classify a depression level and normality state of a target patient by applying variable data for each nutritional factor obtained for the target patient to a pre-learned depression level prediction model (S320).

Next, the depression prediction device 100 can output and provide the classification result through the user terminal 200 (S330). Here, the classification result may include not only a depression classification class but also a probability value for each class.

Furthermore, the depression prediction device 100 can analyze the variable data for each nutritional factor of the target patient input to the depression level prediction model, and may present analysis content of impact for each factor on which nutritional factor causes the classification result of the depression level for a corresponding patient to be derived, by using explainable artificial intelligence, and accordingly, the depression prediction device 100 can assist in identifying the cause of depression diagnosis and determining a treatment scope.

Also, in step S310, the depression prediction device 100 can further obtain demographic information including gender and age of the target patient, and in step S320, the depression prediction device 100 can also determine differently combinations of nutritional factors applied to the depression level prediction model based on the gender and age of the target patient and classify a severity depression level and a normality state.

The following describes a specific example of a learning process of a prediction model. The learning unit can construct learning data for model learning.

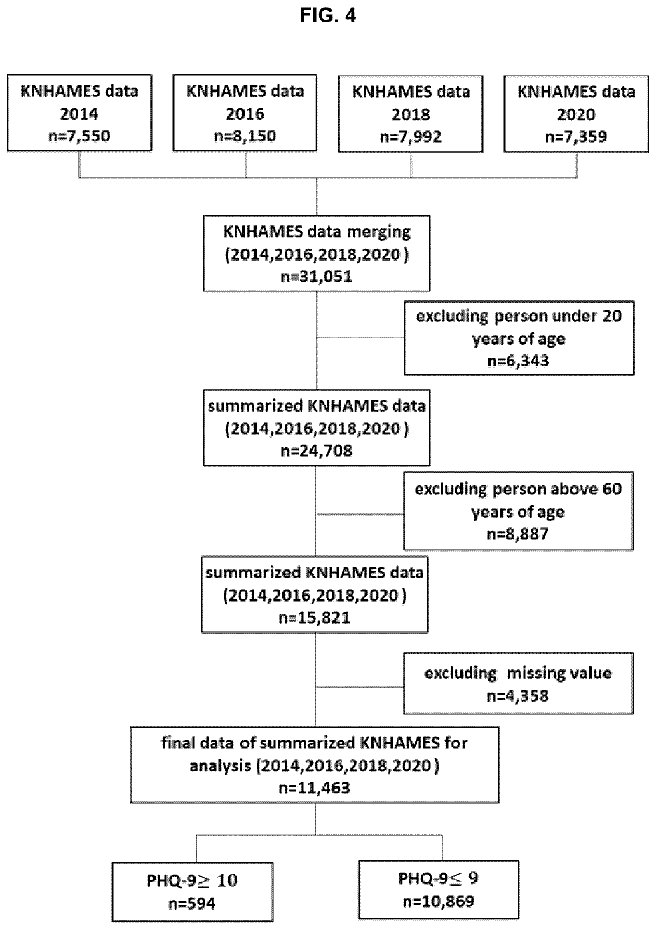

FIG. 4 is an example diagram illustrating a learning data construction process according to an embodiment of the present invention. FIG. 4 illustrates learning data constructed by filtering male and female target persons aged 20 to 59 years from among target persons whose PHQ-9 and nutrient intake amount variables are measured in each year (2014, 2016, 2018, and 2020) in the Korea national health and nutrition examination survey.

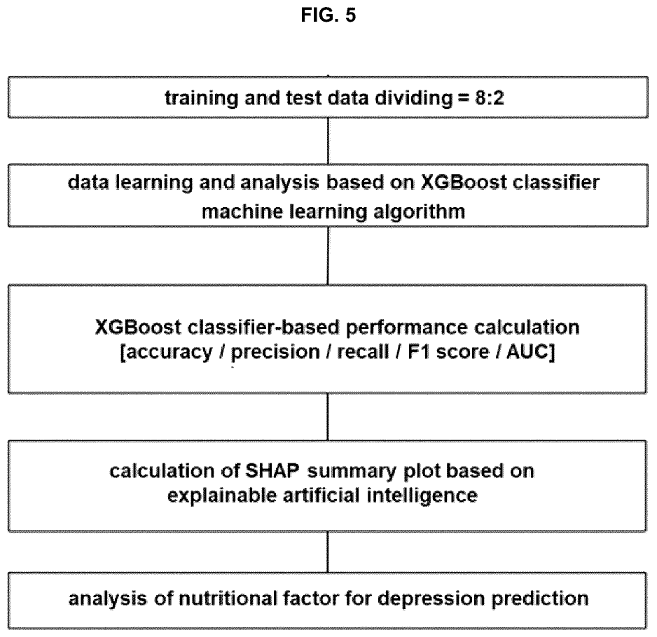

FIG. 5 is an example diagram illustrating a depression prediction model learning and interpretation process considering nutritional factors in an embodiment of the present invention.

The learning data can be divided into a learning set and a test set for artificial intelligence learning. In an embodiment of the present invention, the learning set and the test set were divided in an 8:2 ratio and data was trained by an XGBoost classifier to obtain a depression model considering nutritional factors. In order to determine classification accuracy, accuracy, precision, recall, F1 score (Fi score), and AUC (Area Under the Curve) were calculated.

In addition, based on explainable artificial intelligence, SHAP summary plots for four groups, which are categorized by gender (male/female) and age (20 to 39/40 to 59), were calculated and the analysis results were interpreted. The XGBoost was used because this algorithm demonstrated strong overall performance and, at the same time, identified a key research result through analysis result summary plots according to SHAP (SHapley Additive explanation) values. Table 1 below shows an accuracy result of a depression prediction model considering nutritional factors.

TABLE 1

| Group | Accuracy | Precision | Recall | F1 score | AUC |
|---|---|---|---|---|---|
| 20-39, Male | 95.3 | 92.9 | 95.3 | 93.6 | 0.83 |
| 20-39, Female | 92.3 | 88.9 | 92.3 | 90.1 | 0.80 |
| 40-64, Male | 97.9 | 97.5 | 97.9 | 97.3 | 0.84 |
| 40-64, Female | 93.8 | 91.8 | 93.8 | 92.0 | 0.88 |
| Total | 94.3 | 92.6 | 94.3 | 93.1 | 0.88 |

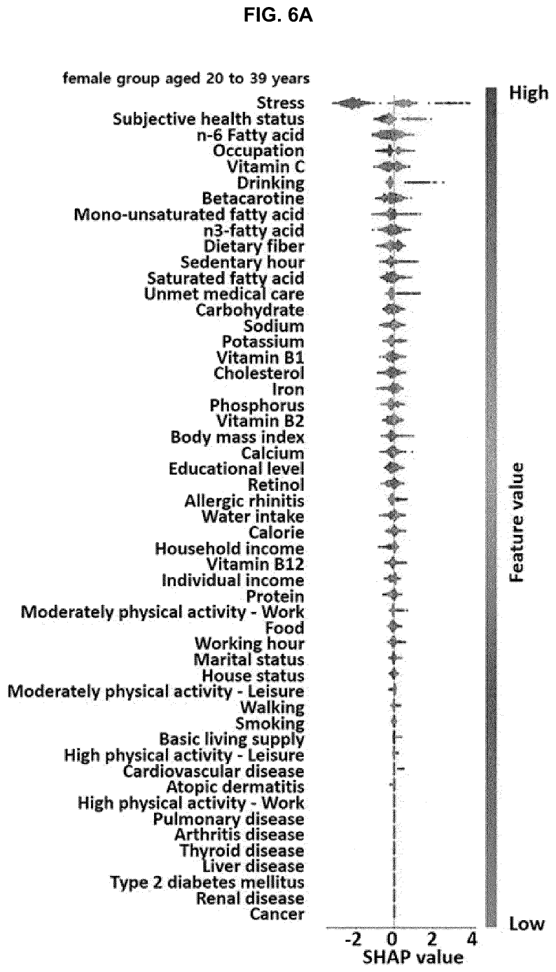

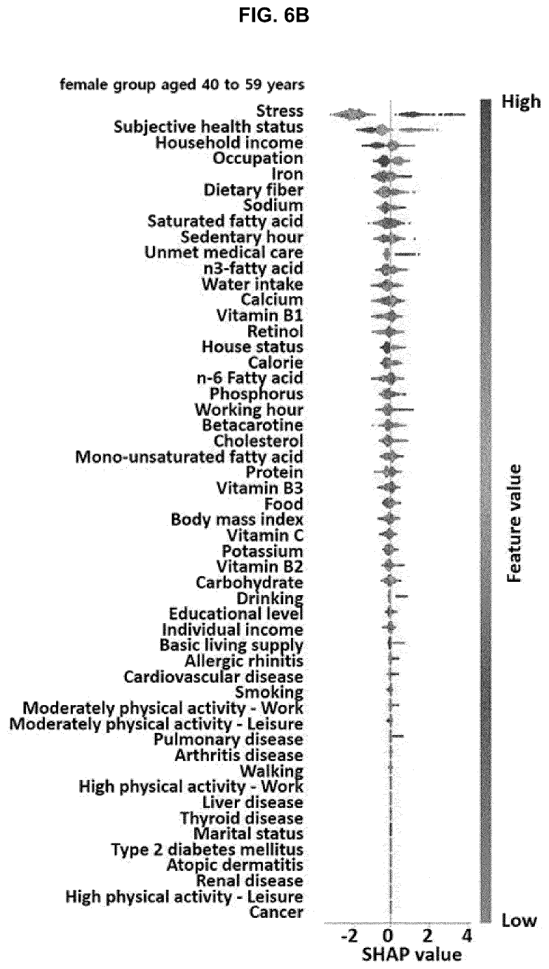

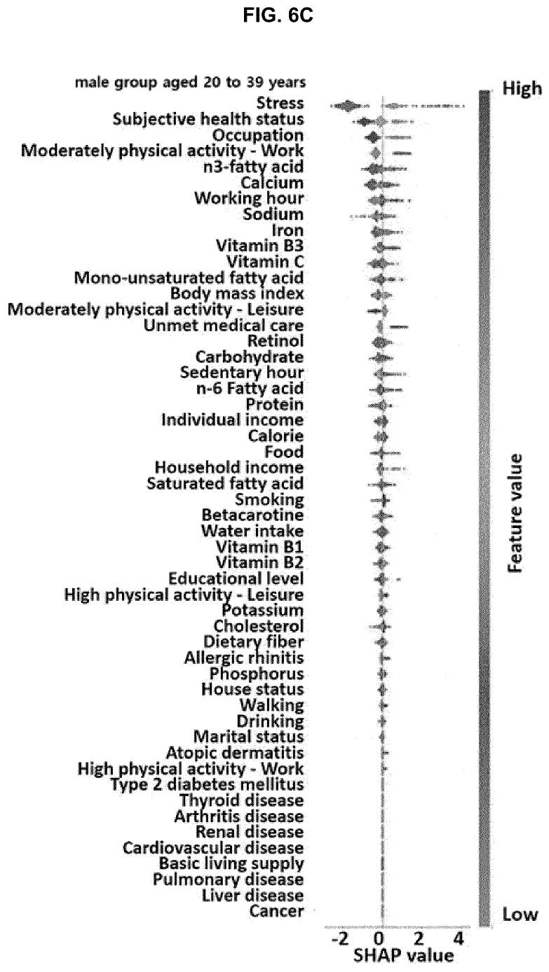

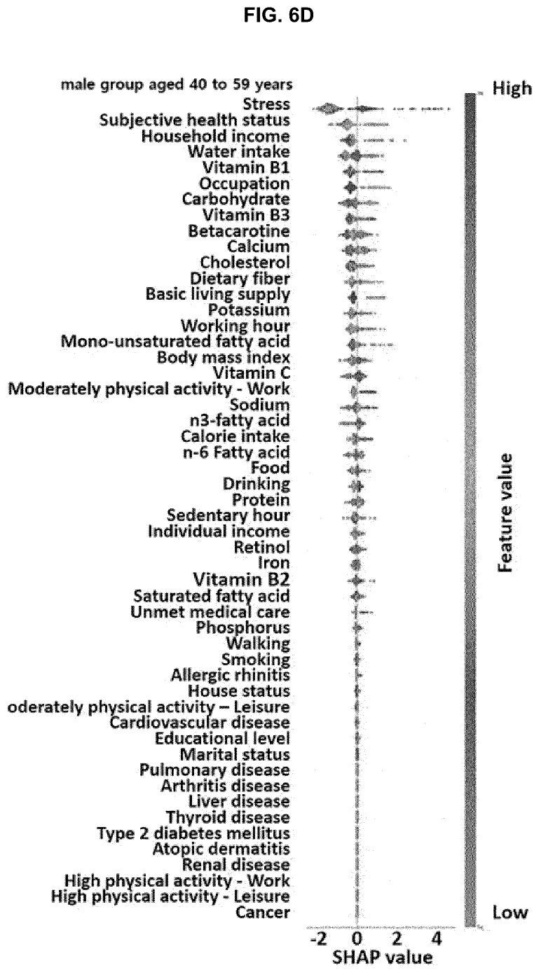

FIGS. 6A to 6D are example diagrams illustrating depression prediction results considering nutritional factors to which differences in gender and age are reflected in an embodiment of the present invention. From the results, for adult males aged 20 to 39 years, n3-fatty acid, calcium, sodium, iron, vitamin B3, vitamin C, monounsaturated fatty acid, retinol, carbohydrate, and n6-fatty acid are found to be top 10 priority nutrients for predicting depression in this order, and for adult males aged 40 to 59 years, a water intake amount, vitamin B1, carbohydrate, vitamin B3, beta-carotene, calcium, cholesterol, dietary fiber, potassium, and monounsaturated fatty acid are found to be top 10 priority nutrients for predicting depression in this order.

Furthermore, for adult females aged 20 to 39 years, n6-fatty acid, vitamin C, beta-carotene, monounsaturated fatty acid, n3-fatty acid, dietary fiber, saturated fatty acid, carbohydrate, sodium, and potassium are found to be top 10 priority nutrients for predicting depression in this order, and for adult females aged 40 to 59 years, iron, dietary fiber, sodium, saturated fatty acid, n3-fatty acid, water, calcium, vitamin B1, retinol, and n6-fatty acid are found to be top 10 priority nutrients for predicting depression in this order.

As such, the present invention can apply a methodology for interpreting machine learning models, such as SHAP, to evaluate contribution and importance of each feature (input variable) with respect to the prediction, enabling interpretation of prediction results and providing an importance assessment result for each interpreted input variable.

According to the present invention, by applying nutritional factors considering a patient's dietary state to an artificial intelligence model, depression levels and normality states can be predicted without the need for a medical professional's direct assessment.

Furthermore, the present invention predicts depression through nutritional factors, which are common elements essential for human health worldwide, and thus, the present invention can be used and applied internationally and can also be utilized in the development of health functional foods that can help prevent and treat depression, as well as in personalized precision medicine and diet-related digital healthcare services.

While the present invention is described with reference to the embodiments illustrated in the drawings, these are merely examples, and those skilled in the art will understand that various modifications and equivalent alternative embodiments can be derived therefrom. Therefore, the true scope of the present invention should be defined by the technical idea of the appended claims.

What is claimed is:

1. A depression prediction device based on nutritional factors, the depression prediction device comprising:
a data collection unit configured to obtain variable data for each of multiple nutritional factors analyzed from a dietary status of a target patient; and
a classification unit configured to classify a depression level and a normality state of the target patient by inputting the variable data for each of the multiple nutritional factors to a pre-learned depression level prediction model.

2. The depression prediction device of claim 1, wherein the classification unit classifies the depression level of the target patient into one of a moderate level, a mild level, and a normal level.

3. The depression prediction device of claim 1, wherein the data collection unit generates variable data for each nutritional factor based on a nutrient intake state analyzed from a diet history of the target patient during a set period.

4. The depression prediction device of claim 1, wherein the multiple nutritional factors include a plurality of factors selected from n3-fatty acid, calcium, potassium, sodium, iron, vitamin B1, vitamin B3, vitamin C, monounsaturated fatty acid, retinol, carbohydrate, n3-fatty acid, n6-fatty acid, a water intake amount, cholesterol, dietary fiber, monounsaturated fatty acid, and saturated fatty acid.

5. The depression prediction device of claim 1, further comprising:
a provision unit configured to analyze the variable data for each of the multiple nutritional factors of the target patient input to the depression level prediction model, and presents analysis content of impact for each factor on which nutritional factor causes a classification result of a depression level for a corresponding patient to be derived, by using explainable artificial intelligence.

6. The depression prediction device of claim 1, wherein
the data collection unit further obtains demographic information including gender and age of the target patient, and
the classification unit classifies a depression severity level and a normal state by determining differently a combination of nutritional factors to be applied to the depression level prediction model depending on the gender and age of the target patient.

7. The depression prediction device of claim 6, wherein
the classification unit classifies a depression level and a normality state of a target patient who is male and aged between 20 and 39 years by utilizing variable values of factors corresponding to n3-fatty acid, calcium, sodium, iron, vitamin B3, vitamin C, monounsaturated fatty acid, retinol, carbohydrate, and n6-fatty acid among variable values for each nutritional factor collected for the target patient, and
the classification unit classifies a depression level and a normality state of a target patient who is female and aged between 20 and 39 years by utilizing variable values of factors corresponding to n6-fatty acid, vitamin C, beta-carotene, monounsaturated fatty acid, n3-fatty acid, dietary fiber, saturated fatty acid, carbohydrate, sodium, and potassium among variable values for each nutritional factor collected for the target patient.

8. The depression prediction device of claim 7, wherein
the classification unit classifies a depression level and a normality state of a target patient who is male and aged between 40 and 59 years by utilizing variable values of factors corresponding to a water intake amount, vitamin B1, carbohydrate, vitamin B3, beta-carotene, calcium, cholesterol, dietary fiber, potassium, and monounsaturated fatty acid among variable values for each nutritional factor collected for the target patient, and
the classification unit classifies a depression level and a normality state of a target patient who is female and aged between 40 and 59 years by utilizing variable values of factors corresponding to iron, dietary fiber, sodium, saturated fatty acid, n3-fatty acid, a water intake amount, calcium, vitamin B1, retinol, and n6-fatty acid among variable values for each nutritional factor collected for the target patient.

9. A depression prediction method performed by a depression prediction device based on nutritional factors, the depression prediction method comprising:
a step of obtaining variable data for each of multiple nutritional factors analyzed from a dietary status of a target patient; and
a step of classifying a depression level and a normality state of the target patient by inputting the variable data for each of the multiple nutritional factors to a pre-learned depression level prediction model.

10. The depression prediction method of claim 9, wherein in the step of classifying, the depression level of the target patient is classified into one of a moderate level, a mild level, and a normal level.

11. The depression prediction method of claim 9, wherein in the step of obtaining, the variable data for each nutritional factor is generated based on a nutrient intake state analyzed from a diet history of the target patient during a set period.

12. The depression prediction method of claim 9, wherein the multiple nutritional factors include a plurality of factors selected from n3-fatty acid, calcium, potassium, sodium, iron, vitamin B1, vitamin B3, vitamin C, monounsaturated fatty acid, retinol, carbohydrate, n3-fatty acid, n6-fatty acid, a water intake amount, cholesterol, dietary fiber, monounsaturated fatty acid, and saturated fatty acid.

13. The depression prediction method of claim 9, further comprising:
a step of analyzing the variable data for each of the multiple nutritional factors of the target patient input to the depression level prediction model, and presenting analysis content of impact for each factor on which nutritional factor causes a classification result of a depression level for a corresponding patient to be derived, by using explainable artificial intelligence.

14. The depression prediction method of claim 9, wherein
in the step of obtaining, demographic information including gender and age of the target patient is further obtained, and
in the step of classifying, a depression severity level and a normal state are classified by determining differently a combination of nutritional factors to be applied to the depression level prediction model depending on the gender and age of the target patient.
